(19) United States
(12) Patent Application Publication
(10) Pub. No.: US 2009/0148644 A1
(43) Pub. Date: Jun. 11, 2009

(54) HIGHLY CONFORMABLE ADHESIVE DEVICE FOR COMPOUND, MOVING OR VARIABLE FORMS

(76) Inventor: Michael David Francis, Martinsville, IN (US)

Correspondence Address: Michael David Francis, 4098 North Foxcliff Drive East, Martinsville, IN, 46151, US

(21) Appl. No.: 12/239,721

(22) Filed: Sep. 26, 2008

Related U.S. Application Data
(60) Provisional application No. 60/978,271, filed on Oct. 8, 2007.

(51) Int. Cl. C09J 7/04; A61L 15/16

(52) U.S. Cl. 428/41.8; 442/38; 428/193; 428/43

(57) ABSTRACT

An adhesive device for compound or moving forms includes a pliable backing layer or shell. A cloth scrim including interlocking fibers oriented to provides elasticity in at least one direction. The interlocking fibers are straight and are arranged in a bias weave or the interlocking fibers are knitted or woven circular rings, and provide elasticity in multiple directions. The cloth scrim is laminated to the pliable backing layer or shell. An adhesive coating is applied to the cloth scrim for adhering the adhesive device to the compound or moving forms where the elasticity of the cloth scrim allows the adhesive device to conform to the compound or moving forms while minimizing wrinkling or puckering of the adhesive device. The edges of the cloth scrim laminated to the shell or pliable backing layer are pinked to provide increased linear contact at the edges thereby reducing de-lamination of the adhesive device from the surfaces of the compound or moving forms, and further minimizing wrinkling or puckering of the adhesive device. Such characteristics are particularly desirable in applications where intimate and very close contact between the adhesive device and the compound or moving form is crucial to the performance of the device for example in Transdermal Drug Delivery Systems (Skin Patches)

### CROSS-REFERENCE TO RELATED APPLICATIONS

The present Utility patent application claims priority benefit of the U.S. provisional application for patent serial number 60978271 filed on Oct. 8, 2007 under 35 U.S.C. 119(e). The contents of this related provisional application are incorporated herein by reference for all purposes.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to cloth adhesive tapes and other adhesive devices. More particularly, the invention relates to highly conformable and elastic adhesive tape or other adhesive devices.

### BACKGROUND OF THE INVENTION

Duct tape is a cloth tape typically comprising a fabric scrim coated with a polyethylene resin on one side and an acrylic or natural rubber based adhesive on the other side. In addition, other types of adhesive tape exist that are constructed in this manner with a cloth or fabric layer. Unlike single laminar tapes, the fabric scrim gives these cloth tapes strength, yet allows the cloth tapes to be easily torn by hand, transversely or longitudinally. Cloth adhesive tape is also moderately conformable, though not greatly so, and can adhere to a wide variety of surfaces. While duct tape was primarily designed for air ducts and similar commercial applications, consumers have found a broad range of uses for this popular product. Cloth adhesive tape can be used for any number of household repair jobs, such as, but not limited to, as a fastener and in car maintenance. In addition, there are numerous military and aerospace applications for cloth adhesive tape. Other applications of cloth adhesive tape or other adhesive devices where conformability, elasticity, the ability to wear the device, mobility and good edge adhesion properties are desirable include, without limitation, wire harness wrapping tape, transdermal drug delivery systems substrates (i.e., skin patches), sanitary napkins, diapers, and adhesive bandages

While in a simple form, an adhesive tape may consist of a layer of adhesive coated onto a backing material (commonly known to those skilled in the art as a ‘shell’) in what may be called a two-layer structure; however, these adhesive tapes should typically be strong as well as flexible. Consequently, an intermediate cloth layer, or scrim, is sandwiched between the adhesive layer and backing or shell for increased strength in typical commercially available duct tapes. An ultimate tensile strength of many Newtons per square meter (N/m²) or pounds-force per square inch (lbf/in²) is claimed by currently known cloth adhesive tapes, although practical applications of such moderate tensile strength are limited. In such commercially available prior art cloth adhesive tapes, the adhesive layer is typically a per se known pressure-sensitive adhesive, for example, an acrylic or rubber-based pressure-sensitive adhesive, and the backing or shell comprises a polyolefin, for example, polyethylene.

Cloth adhesive tapes of this description are generally satisfactory for their intended usage and accordingly have achieved wide market acceptance. Nevertheless, it will be appreciated that, like any commercially available product, there is always a great need for functional improvement. For example, without limitation, there is a need for improved techniques for providing a cloth adhesive tape that is highly conformable and elastic. Furthermore, there is a need for a cloth adhesive tape that provides enhanced edge adhesion and that resists wrinkling or puckering, which can lead to loss of intimate contact, where such a feature is considered desirable (for example in Transdermal Drug Delivery Systems—skin patches)

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary piece of woven scrim, in accordance with the prior art;

FIGS. 2A and 2B illustrate exemplary woven scrim cloths. FIG. 2A illustrates an exemplary conventional weave scrim cloth, in accordance with the prior art, and FIG. 2B illustrates an exemplary bias weave scrim cloth, in accordance with an embodiment of the present invention;

FIG. 3 illustrates an exemplary knitted ring scrim for a cloth adhesive tape or device, in accordance with an embodiment of the present invention;

FIG. 4 illustrates an exemplary piece of adhesive tape with pinked edges, in accordance with an embodiment of the present invention;

FIGS. 5A and 5B illustrate an exemplary piece of adhesive tape, in accordance with an embodiment of the present invention. FIG. 5A is a top view with the various layers the tape shown peeled away from a backing layer or shell for clarity, and FIG. 5B is a cross sectional view;

FIG. 6 illustrates exemplary finished width strips cut from a conventional jumbo roll being re-assembled in to one continuous tape, in accordance with an embodiment of the present invention; and

FIG. 7 illustrates an exemplary piece of adhesive tape with exemplary semi-penetration perforation designs, in accordance with an embodiment of the present invention.

Unless otherwise indicated illustrations in the figures are not necessarily drawn to scale.

### SUMMARY OF THE INVENTION

To achieve the forgoing and other objects and in accordance with the purpose of the invention, a highly conformable cloth adhesive device is presented.

In one embodiment, a highly conformable adhesive device for application to variable, moving or compound forms is presented. The adhesive device includes a pliable backing layer or shell. A cloth scrim including interlocking fibers is oriented to provides elasticity in at least one direction. The cloth scrim is laminated to the pliable backing layer or shell. An adhesive coating is applied to the cloth scrim for adhering the adhesive device to the compound forms where the elasticity of the cloth scrim allows the adhesive device to conform to the compound or variable forms while minimizing wrinkling or puckering of the adhesive device, and therefore enhancing intimate contact In another embodiment the edges of the cloth scrim laminated to the pliable backing layer are pinked to provide increased area on the edges thereby reducing de-lamination of the adhesive device from surfaces of the compound forms and further minimizing wrinkling of the adhesive device. Another embodiment further includes a release layer applied to the adhesive coating to mitigate the effects of the elasticity until the release layer is removed for adhering the adhesive device to the compound forms. In other various embodiments the interlocking fibers are straight and are arranged in a bias weave, or the interlocking fibers are circular rings, to provide elasticity in multiple directions.

In another embodiment a highly conformable adhesive device for variable or compound forms is presented. The adhesive device includes pliable means for backing or providing a shell for the adhesive device, scrim means for stretching in at least one direction, the scrim means being laminated to the pliable means, and means for adhering the adhesive device to the compound or variable forms where the stretching of the scrim means allows the adhesive device to conform to the compound or variable forms while minimizing wrinkling or puckering of the adhesive device. Various other embodiments further include means for mitigating the effects of the stretching until the mitigating means is removed for adhering the adhesive device to the compound or variable forms and means for facilitating hand tearing of the adhesive device, should that be a requirement of the devices application.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention will now be described in detail with reference to embodiments thereof as illustrated in the accompanying drawings.

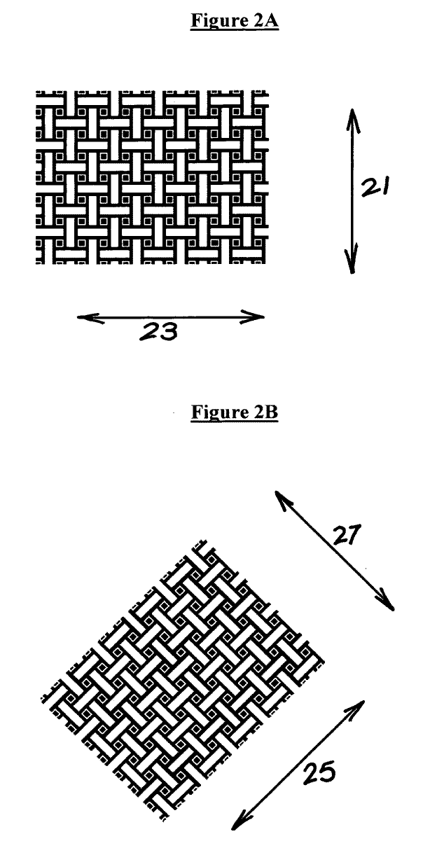

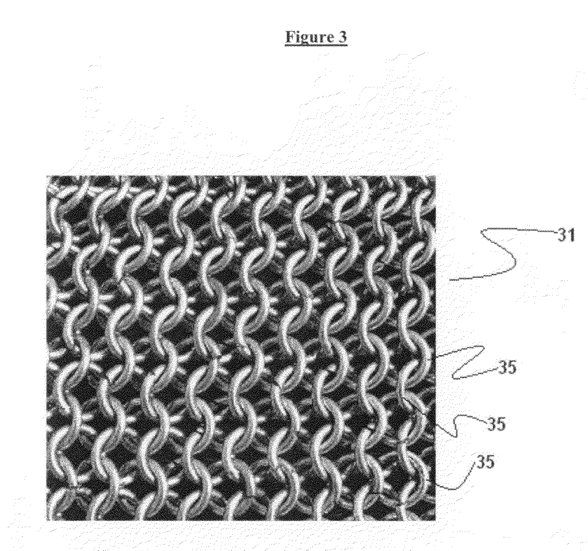

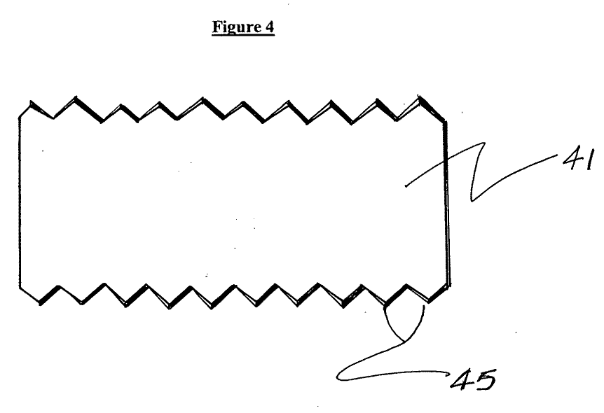

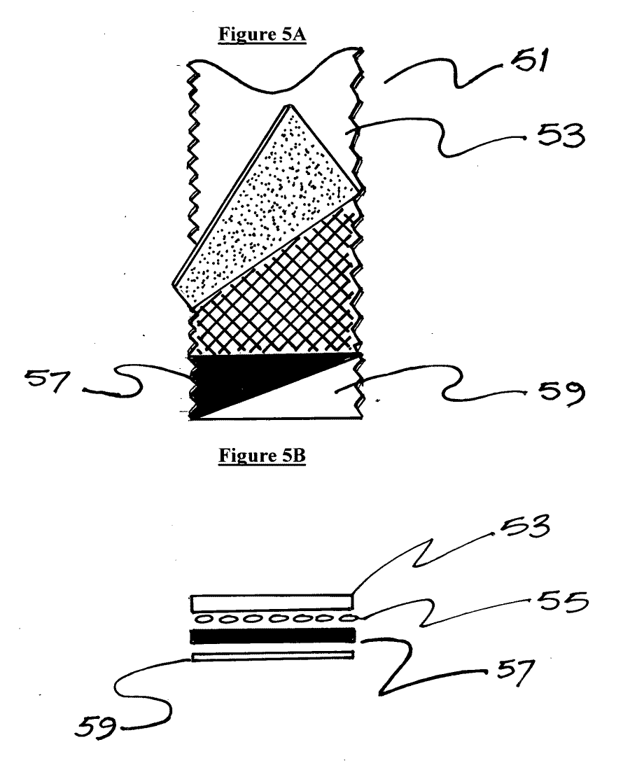

Disclosed herein is a novel laminar tape structure in which either a bias-weave cloth scrim (yarns for which are arranged diagonally to the longitudinal edge—FIG. 2B), or a knitted textile mesh comprising of multiple interlocking circular rings (hereinafter referred to as ‘knitted ring’) (FIG. 3) is employed. The bias-weave or knitted ring cloth scrim FIG. 5—55 is sandwiched between an outer backing element or shell FIG. 5—53 and an adhesive layer (FIG. 5—57). The tape is additionally manufactured with ‘pinked’ or scalloped longitudinal edges (FIG. 4\textcircled{6}). Such arrangement shall permit taping of curved, irregular, moving, variable or uneven surfaces, where conventional duct tapes would not be so conformable, or retain their edge adhesion. The bias weave or knitted ring of the backing material or shell and the pinked edges of the tape work together to create effective stretchability, elasticity and conformance around variable or compound forms. This is of particular importance in applications such as in Medical Devices, First Aid or Personal Hygiene products where the product is ‘wearable’ and must maintain intimate contact with the human form. Such practical applications may be (but not limited to) diapers, sanitary napkins/towels and Transdermal Drug Delivery Systems (otherwise known as ‘skin patches’)

Also disclosed herein is a description of a method of providing such an improved adhesive tape or device. In considering how to solve this task, the individual components or elements which constitute the product were considered and it was concluded that improvement in four areas could be made, namely a) conformity to curved, irregular, moving or uneven surfaces b) providing applied tensional, elastic and stretch support, c) increasing the contact area of the longitudinal (Machine Direction) tape edge, thereby reducing de-laminating, or the tendency to curl away from the edges and d) in one of the preferred embodiments, bias weave and knitted ring cloth scrims are difficult to handle during the manufacturing phase, not least because the very properties which are preferable in an elastic tape are prone to causing deformation and stretch during manufacture and subsequent usage, so a method has been developed which eliminates this characteristic until such time as it is required during actual finished use. The method employed requires that a removable peel-ply or release backing remains in contact with the tape, until final application by the user. In practice, the tape could be applied with a proprietary tape dispenser, such as that described in U.S. Pat. No. 5,468,332 Dretzka et al.

Consequently, in the one embodiment fully described, attention was focused on the open weave cloth scrim layer, the edge finishing, and the final finishing and processing of the jumbo roll.

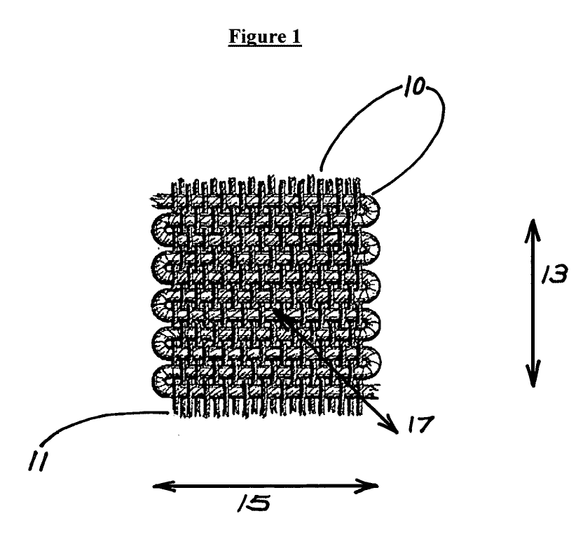

In accordance with the present disclosure, the task is solved in an elegant manner by substituting the conventional scrim material (the yarns for which are, in prior art, arranged perpendicular and parallel to the longitudinal edge—FIG. 1—10) with a scrim material the yarns for which are arranged in a bias-weave fashion i.e. diagonally to the longitudinal edge (FIG. 2—25 and —27), or in a multiple interlocking textile ring fashion (FIG. 3). This provides the tape or device with inherent stretch, conformability, elasticity and applied tension when so deployed. The resultant tape, when stretched around a compound or variable curve, irregular, moving or uneven surface will do so with greater conformity and tension than tapes currently known to those practiced in the art. The conformance mechanism is well understood, in that the tape will narrow in width, by virtue of the bias weave or knitted rings imparting compressional transverse force, as longitudinal tension force is applied, this narrowing in width under tension providing elastic or conformable properties.

Additionally, the longitudinal (Machine Direction) edge of the tape, by virtue of the ‘pinking’ or ‘scalloping’ is provided with over 40% more linear contact for edge adhesion, thereby reducing de-laminating of the tape from the surface, or the tendency to curl away from the edges (FIG. 4-45).

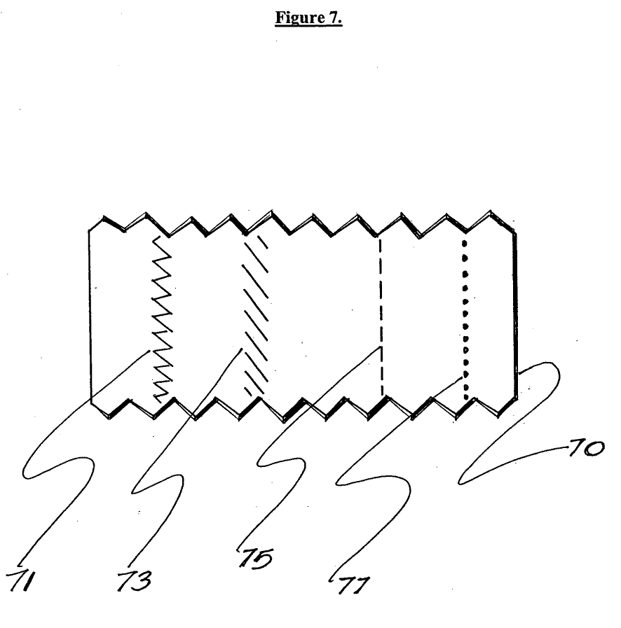

The tape, or device in at least one embodiment, may still be hand-tearable. This is achieved by perforating the tape by partial-penetration Slitting (in the shape of chevrons, lines or dots) in the Cross Direction (see FIG. 7), thus weakening the scrim where so perforated.

Preferred embodiments of the present invention provide a cost effective laminar, adhesive tape or other adhesive device having a bias weave or knitted ring cloth scrim layer for reinforcement and increased conformability. In preferred embodiments a bias weave cloth scrim comprises yarns that are arranged diagonally to the longitudinal edge of the adhesive tape or device, as shown by way of example in FIG. 2B, and a knitted ring cloth scrim comprises multiple interlocking circular rings of yarn, as shown by way of example in FIG. 3. In preferred embodiments, the bias weave or knitted ring cloth scrim is sandwiched between an outer backing element (or shell) and an adhesive layer, as shown by way of example in FIGS. 5A and 5B. This provides an adhesive tape or device with inherent stretch, elasticity, non-wrinkling, non-puckering properties, and applied tension. The resultant adhesive tape or device in preferred embodiments, when stretched around a compound curve, irregular, moving or uneven surface does so with greater conformity and tension than cloth adhesive tapes currently known to those skilled in the art. The conformance mechanism is well understood, in that the tape narrows in width by virtue of the bias weave or knitted ring scrim imparting compressional transverse force as longitudinal tension force is applied; this narrowing in width under tension provides elastic properties.

In some embodiments, the adhesive tape or device is additionally manufactured with pinked or scalloped longitudinal edges, as shown by way of example in FIG. 4. These embodiments more easily enable taping of curved, irregular, moving or uneven surfaces, where conventional cloth adhesive tapes are not so conformable and may not retain their edge adhesion. The bias weave or knitted ring of the backing material and the pinked edges of the tape work together to create effective elasticity, reduced wrinkling and puckering and provide conformance around compound shapes or around the moving human form, in the case of medical devices, first aid or personal hygiene products. This is of particular importance in applications where the adhesive device is ‘wearable’ and must maintain intimate contact with the human form, for example, without limitation, diapers, sanitary napkins/towels, and Transdermal Drug Delivery Systems (i.e., skin patches).

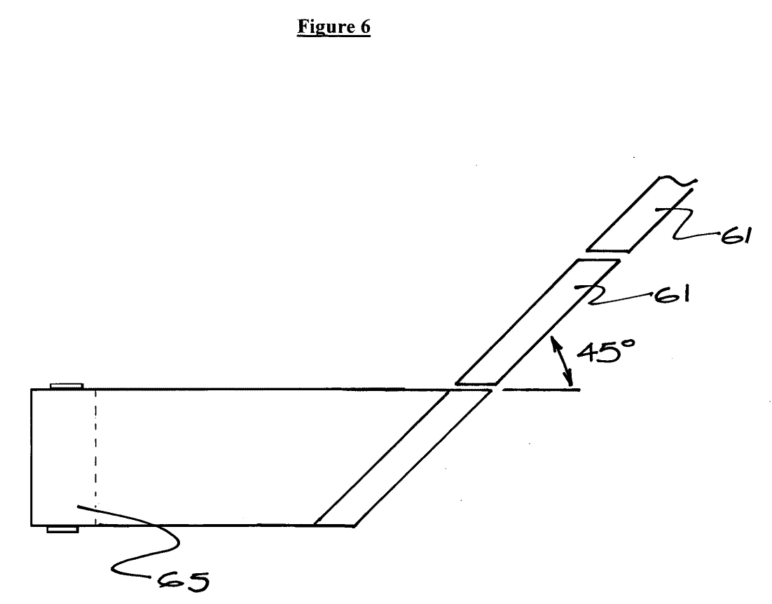

As described in further detail below, at least one embodiment of a method of manufacturing such tape is described comprising the processing of conventional jumbo rolls (FIG. 6—65) whose warp and weft threads are parallel or perpendicular to the machine edge into tapes that have a bias weave whose warp and weft threads are diagonal in relation to the machine edge (FIG. 6—61).

FIG. 1 illustrates an exemplary piece of woven scrim, in accordance with the prior art. In its simplest form, cloth adhesive tape comprises a cloth material, or woven scrim, carrying an adhesive layer. Referring to FIG. 1, in conventional woven scrim, yarns 10 are arranged to be perpendicular and parallel to a longitudinal edge 11, which is parallel to the machine direction of the scrim, indicated in the Figure by an arrow 13. An arrow 15 indicates the cross direction of the scrim, which is perpendicular to the machine direction. An arrow 17 illustrates the bias cross direction (BCD) of the scrim, which is at a forty-five degree angle to the machine direction.

FIGS. 2A and 2B illustrate exemplary woven scrim cloths. FIG. 2A illustrates an exemplary conventional weave scrim cloth, in accordance with the prior art, and FIG. 2B illustrates an exemplary bias weave scrim cloth, in accordance with an embodiment of the present invention. Woven cloths have a warp and a weft. Referring to FIG. 2A, in the conventional weave scrim cloth the warp is parallel to the machine direction as indicated by an arrow 21, and the weft is perpendicular to the machine direction as indicated by an arrow 23. Referring to FIG. 2B, in the scrim employed in the present embodiment the warp and weft, indicated by arrows 25 and 27, respectively, are at forty-five degree angles to the machine direction of the scrim.

FIG. 3 illustrates an exemplary knitted ring scrim 31 for a cloth adhesive tape or device, in accordance with an embodiment of the present invention. In the present embodiment, knitted ring scrim 31 comprises multiple knitted textile interlocking rings 35. Knitted ring scrim 31 is highly conformable and may easily be worn on parts of the human body. Rings 35 in knitted ring scrim 31 may be made of various materials including, but not limited to, cotton or man-made fibers.

FIG. 4 illustrates an exemplary piece of adhesive tape 41 with pinked edges 45, in accordance with an embodiment of the present invention. In the present embodiment, tape 41 comprises pinked or scalloped longitudinal edges 45 in the machine direction. Pinked edges 45 more easily enable the taping of curved, irregular, moving or uneven surfaces, where conventional cloth adhesive tapes would not be so conformable or retain their edge adhesion. Pinked edges 45 provide over 40% more linear contact for edge adhesion, thereby reducing de-laminating of tape 41 from the surface to which tape 41 is applied and the tendency of tape 41 to curl away from the edges. In the present embodiment, adhesive tape 41 may comprise scrim of either a bias weave or a knitted ring weave. Alternate embodiments of the present invention may implement pinking or scalloping that is shaped differently, for example, without limitation, a rounded scallop or a square notch, and other alternate embodiments may be implemented without pinked or scalloped edges.

FIGS. 5A and 5B illustrate an exemplary piece of adhesive tape 51, in accordance with an embodiment of the present invention. FIG. 5A is a top view with the various layers of tape 51 shown peeled away from a backing layer or shell 53 for clarity, and FIG. 5B is a cross sectional view. In the present embodiment, adhesive tape 51 comprises a laminar structure comprising a pliable outer layer 53 comprising a polymeric coating, a bias weave or knitted ring cloth scrim 55 or other reinforcing fabric, a layer of adhesive 57, for example, without limitation, a per se known pressure-sensitive adhesive, and a temporary peel-ply or release backing layer 59.

In the present embodiment, pliable outer layer or shell 53 is laminated to scrim 55. The pliable outer layer 53 may be, for example, without limitation, on the order of 4-6 thousandths of an inch thick and may be made of a per se known flexible plastic support material.

In the present embodiment, scrim 55 is preferably a woven or knitted web that enables a user to easily tear the tape in the cross (CD) direction, for example, without limitation, the CD illustrated by way of example with arrow 15 in FIG. 1, as distinguished from non-woven cloth, which does not have such hand-tearing characteristics. Scrim 55 is preferably a bias weave scrim as shown by way of example in FIG. 2B or a knitted ring scrim as shown by way of example in FIG. 3. Useful woven webs of this description may be made from various types of natural and synthetic fibers such as, but not limited to, those heretofore employed in the manufacture of cloth adhesive tapes. Scrim 55 may, for example, without limitation, be on the order of 2-8 thousandths of an inch thick. For flexibility as well as the ability to manufacture the tape in roll form, scrim 55 ordinarily should not have a dense thread count.

While in theory adhesive 57 may comprise a per se known heat or water-activated adhesive, for ease of application by the user adhesive 57 is preferably a known pressure-sensitive adhesive. Useful adhesives for this purpose include, without limitation, acrylic or rubber based adhesive formulations. As will be well understood by those skilled in the art, such formulations also typically contain tackifiers, fillers and other components performing specific functions. Selection of a suitable adhesive for adhesive 57 will be a matter of individual choice within the expected judgment of the skilled worker. In the present embodiment, adhesive 57 may be, for example, without limitation, on the order of 3-6 thousandths of an inch thick and, as previously stated, it is preferably a per se known pressure-sensitive adhesive, for example, without limitation, a rubber-based or acrylic adhesive formulation.

In order to facilitate the rewinding of the finished tape onto final rolls and to protect the adhesive loaded pinked edges, removable peel-ply or release backing 59 is applied to adhesive 57 of the master roll, after the adhesive coating operation and prior to the cutting operation.

In the present embodiment, tape 51 can be assembled by individually producing and sequentially coating the components parts. For example, without limitation, pliable outer or shell layer 53 and scrim 55 may be individually supplied to a calendering operation to provide a laminar structure of these two components. Adhesive 57 may then be coated onto the cloth scrim substrate by known coating techniques. Peel-ply or release backing layer 59 is also applied to adhesive 57 by known web application techniques.

Disclosed herein is an exemplary method of manufacturing an adhesive tape or other adhesive device that comprises a bias weave scrim cloth. Bias weave cloth scrims are difficult to handle during the manufacturing phase, not least because the very properties that are preferable in an elastic tape are prone to causing deformation and stretch during manufacture and subsequent usage. Therefore the following method generally eliminates this characteristic of elasticity until such time as it is required during actual finished use. The method illustrated by way of example in accordance with FIG. 6 requires that a removable peel-ply or release backing layer remains in contact with the tape until final application by the user.

FIG. 6 illustrates exemplary finished width strips 61 cut from a conventional jumbo roll 65 being re-assembled in to one continuous tape, in accordance with an embodiment of the present invention. In the present embodiment, strips 61 are cut at intervals equivalent to the forty-five degree hypotenuse of the width of jumbo roll 65. A machine rotary slitting operation performed on jumbo roll 65 to create the pinked or scalloped machine direction edges is well understood by those skilled in the art. Such a slitting operation utilizes a single slit head, or multi slit heads, and, in order to create the bias weave on the finished product, the slitting is performed diagonally to the machine direction. After being cut from jumbo roll 65, strips 61 are welded together with a welded seam to form a continuous tape web with a bias weave. Strips 61 may be re-assembled by thermal or ultrasonic welding. The finished tape has a bias weave. In the present embodiment, the finished tape is wound on to the final rolls with a peel-ply or release backing layer still in place, the peel-ply or release backing layer is removed as the tape is deployed during application by the user. The temporary peel-ply or release backing layer is used for rewinding the final rolls and more easily enables the use of a machine to cut pinked or scalloped machine direction tape edges.

In embodiments of the present invention where a knitted ring backing scrim is employed rather than a bias weave scrim, the manufacture of the adhesive tape or device is a matter of individual choice within the expected judgment of the skilled worker, or those skilled in the art.

FIG. 7 illustrates an exemplary piece of adhesive tape 70 with exemplary semi-penetration perforation designs, in accordance with an embodiment of the present invention. In the present embodiment, various perforation designs are shown including a chevron 71, a partial-chevron 73, a line 75, and a dot pattern 77. These semi-penetration perforation designs facilitate hand tearing of tape 70 by weakening the scrim where so perforated. The cloth scrim, in at least one embodiment, is a woven web that enables easy hand tearing in the cross direction (CD). In these embodiments and in embodiments where the ability to hand tear the tape is not necessary, semi-penetration perforations may not be included.

As stated previously, while a polymeric is the preferred outer layer or shell material, other materials may be employed in alternate embodiments. In the selection of suitable materials, in general factors such as, but not limited to, cost, flexibility, cohesion, sterility, water resistance, UV resistance, non-allergenic properties, anerobic properties and compatibility are to be considered. In any event, the substitution of other materials for the polymeric in the illustrative embodiment is a matter of individual choice within the expected judgment of the skilled worker, as will be the particular thicknesses of the backing materials and/or adhesives.

In some embodiments of the present invention, the bias weave or knitted ring scrim substrate and pinked edges may be applied to products other than adhesive tape that are well known to those skilled in the art, such as, but not limited to, wire harness wrapping tape, Transdermal Drug Delivery Systems substrates (i.e., skin patches), sanitary napkins, adhesive bandages, and diapers.

In typical use of preferred embodiments of the present invention, cloth adhesive tape comprising a bias weave or knitted ring scrim layer is longitudinally elastic and can stretch around a compound curve or an irregular, moving or uneven surface with greater conformity and tension than traditional cloth adhesive tape. The tape in preferred embodiments achieves greater conformity because as the tape stretches longitudinally, it narrows in width by virtue of the bias weave or knitted ring scrim, resulting in a smoother application to a contoured, moving or irregular surface. The characteristics of conformability and adhesion to moving forms (e.g., the human body) are particularly desirable when considering applications for medical devices (e.g., Transdermal Drug Delivery Systems or skin patches), first aid products (e.g., adhesive bandages) and personal hygiene products (e.g., sanitary napkins and diapers).

In one embodiment of the present invention, an adhesive tape comprises an adhesive layer coated onto a backing comprising an open weave cloth scrim material, an outer layer or shell of a polymeric material, and a protective peel-ply or release layer applied during manufacture, the improvement wherein said open weave cloth scrim material is dispositioned in a bias weave (i.e., the warp and weft of the cloth scrim material is diagonal to the longitudinal edge of the tape) or as multiple interlocking knitted rings, thereby being capable of being stretched both multi-directionally.

In another embodiment of the present invention, an adhesive tape comprises an adhesive layer coated onto a backing comprising an open weave cloth scrim material, an outer layer or shell of a polymeric material, and a protective peel-ply or release layer applied during manufacture, the improvement wherein said shell or backing layer, cloth scrim material and adhesive layer has pinked (i.e., scalloped) longitudinal (i.e., machine direction) edges.

In one embodiment, a method of manufacturing an adhesive tape is provided wherein a conventionally manufactured master roll (i.e., a jumbo roll) of non-bias weave tape (i.e., the warp and weft of the cloth scrim are perpendicular or parallel to the machine direction edge) is cut with a pinking slitter or a plurality of pinking slitters diagonally to the machine direction in widths equivalent to the finished tape width.

In a further embodiment, a method of manufacturing an adhesive tape is provided wherein a conventionally manufactured master roll of non-bias weave tape is cut with a pinking slitter or a plurality of pinking slitters diagonally to the machine direction in widths equivalent to the finished tape width, such strips being re-joined with thermal or ultrasonic welding equipment to form a continuous bias weave (i.e., the warp and weft of the cloth scrim are diagonal to the machine direction edge) finished tape.

In a further embodiment, a method of deploying a cloth adhesive tape, known generically as duct tape, is provided wherein the tape may be applied by hand or by a tape dispenser, which facilitates the removal of the peel-ply or release layer for the purposes of convenience and ease of deployment.

In the foregoing description, reference has been made to the use of woven cloth webs because of their ability to be hand torn in the cross direction (CD). If the ability to hand tear the cloth is not a consideration and mechanical severance is instead contemplated and acceptable, it will be appreciated by those skilled in the art that non-woven materials may be employed rather than woven materials. Accordingly, as used in the foregoing and in the appended claims the term “cloth” denotes both woven and non-woven cloth fabrics.

Having fully described at least one embodiment of the present invention, other equivalent or alternative methods of providing a highly conformable and elastic cloth adhesive tape or other adhesive device according to the present invention will be apparent to those skilled in the art. The invention has been described above by way of illustration, and the specific embodiments disclosed are not intended to limit the invention to the particular forms disclosed. For example, the particular implementation of the scrim cloth may vary depending upon the particular type of weaving or knitting technique used. Implementations of the present invention using various weaving and knitting techniques are contemplated as within the scope of the present invention. The invention is thus to cover all modifications, equivalents, and alternatives falling within the spirit and scope of the following claims.

What is claimed is:

1. An adhesive device for compound or moving forms, the adhesive device comprising:
a pliable backing layer or shell;
a cloth scrim comprising interlocking fibers oriented to provide elasticity in at least one direction, said cloth scrim being laminated to said pliable backing layer or shell; and
an adhesive coating applied to said cloth scrim for adhering the adhesive device to the compound or moving forms where said elasticity of said cloth scrim allows the adhesive device to conform to the compound or moving forms while minimizing wrinkling or puckering of the adhesive device.

2. The adhesive device as recited in claim 1, wherein edges of said cloth scrim laminated to said pliable backing layer or shell are pinked to provide increased linear contact at said edges thereby reducing de-lamination of the adhesive device from surfaces of the compound or moving forms and further minimizing wrinkling or puckering of the adhesive device.

3. The adhesive device as recited in claim 2, further comprising a release layer applied to said adhesive coating to mitigate the effects of said elasticity until said release layer is removed for adhering the adhesive device to the compound or moving forms.

4. The adhesive device as recited in claim 2, wherein said interlocking fibers are straight and are arranged in a bias weave.

5. The adhesive device as recited in claim 2, wherein said interlocking fibers are knitted or woven circular rings and provide elasticity in multiple directions.

6. The adhesive device as recited in claim 4, wherein said cloth scrim comprises a woven or knitted web that enables a user to easily tear the adhesive device in a cross direction.

7. The adhesive device as recited in claim 2, wherein said cloth scrim laminated to said pliable backing layer or shell further comprises semi-penetration perforation designs for facilitating hand tearing of the adhesive device by weakening of said cloth scrim where so perforated.

8. The adhesive device as recited in claim 2, wherein the adhesive device is configured as a tape and elasticity is in multiple directions along and across the tape.

9. The adhesive device as recited in claim 2, wherein the adhesive device is configured as a patch, and elasticity is in multiple directions.

10. The adhesive device as recited in claim 2, wherein said adhesive coating comprises a pressure-sensitive adhesive.

11. The adhesive device as recited in claim 2, wherein said pliable backing layer or shell comprises a polymeric material.

12. The adhesive device as recited in claim 8, wherein the tape is further configured on a roll.

13. The adhesive device as recited in claim 9, wherein the patch is further configured for use as a bandage.

14. The adhesive device as recited in claim 9, wherein the patch is further configured for use with Transdermal Drug Delivery Systems Substrates (Skin Patches).

15. An adhesive device for compound or moving forms, the adhesive device comprising:
pliable means for backing or providing a shell for the adhesive device;
scrim means for stretching in at least one direction, said scrim means being laminated to said pliable means; and
means for adhering the adhesive device to the compound or moving forms where said stretching of said scrim means allows the adhesive device to conform to the compound or moving forms while minimizing wrinkling or puckering of the adhesive device.

16. The adhesive device as recited in claim 15, further comprising means for mitigating the effects of said stretching until said mitigating means is removed for adhering the adhesive device to the compound or moving forms.

17. The adhesive device as recited in claim 15, further comprising means for facilitating hand tearing of the adhesive device.
